(19) United States
(12) Patent Application Publication
(10) Pub. No.: US 2014/0194506 A1
(43) Pub. Date: Jul. 10, 2014

(54) Use of Potassium Piperonyl Pentadienoate for Lowering Blood Lipid

(71) Applicants: Gereltu Borjihan, Hohhot (CN); Narisu Bao, Hohhot (CN); Zhaorigetu Sun, Hohhot (CN); Mingfang Wang, Hohhot (CN)

(72) Inventors: Gereltu Borjihan, Hohhot (CN); Narisu Bao, Hohhot (CN); Zhaorigetu Sun, Hohhot (CN); Mingfang Wang, Hohhot (CN)

(73) Assignees: INNER MONGOLIA UNIVERSITY, Hohhot (CN); BORIJIHAN MEDICAL COMPANY OF WUHAN, Hohhot (CN)

(21) Appl. No.: 14/238,333

(22) PCT Filed: Aug. 13, 2012
(86) PCT No.: PCT/CN2012/080025

§ 371 Date: Feb. 11, 2014

(30) Foreign Application Priority Data
Aug. 12, 2011 (CN) 201110230781

(51) Int. Cl. A61K 31/36

(52) U.S. Cl. CPC A61K 31/36
USPC 514/464

(57) ABSTRACT

The invention provides the use of potassium piperonyl pentadienoate for lowering blood lipid, including the use of the potassium piperonyl pentadienoate in the preparation of medicaments and health-care products for lowering serum total cholesterol, lowering low density lipoprotein cholesterol, lowering triglyceride as well as preventing the decrease of high density lipoprotein cholesterol.

### TECHNICAL FIELD

The invention relates to the field of medicinal technology, and particularly, to the use of potassium piperonyl pentadienoate for lowering blood lipid.

### BACKGROUND OF THE RELATED ART

The incidence and mortality of coronary artery disease have increased rapidly in the recent decades around the world. In China, the number of patients associated with hyperlipemia has reached about 200 million. In the whole world, 70 million people die due to the related diseases caused by hyperlipemia every year. Modern medicine researches demonstrate that blood lipid abnormality is closely associated with atherosclerosis (AS) and coronary artery disease (CHD). The use of blood lipid lowering agents is the most efficient medicinal method for treating hyperlipemia and reducing the mortality of heart disease (Shijie Yang, Ruhao Su, et al., Current use and research progress of blood lipid lowering agents, Journal of Medical Forum, 2006, 27(1): 90-92). In order to improve the ability to prevent and treat hyperlipemia and cardio-cerebrovascular diseases in China, research and development of new liquid lowering pharmaceutical compounds have become an important goal for the skilled persons in the art.

The commonly used chemical liquid lowering agents in the world are classified into: (1) statins; (2) fibrates; (3) nicotinic acids; (4) cholic acid chelating (resins) agents; (5) polyenes; etc. The main directions of researches abroad of the blood lipid lowering agents are: (1) the research of cholesterol absorption inhibitors of new types, and its representative drug is Ezetimibe (Zetia); (2) the research of cholesterol ester acyltransferase (ACAT) inhibitors, which develops a new mechanism of treating hypercholesteremia; and (3) the research of new dosage forms and compound recipe drug combinations, such as controlled-release tablets of lovastatin, slow-release formulations of nicotinic acid Nispan, micronized capsule of fenofibrate, the combination of statins and fibrates, etc. In the aspects of chemical drug research, new mechanisms of blood lipid lowering agent are discovered from time to time, as well as a variety of new dosage forms and compound recipe formulations for reducing the adverse effects of drugs and improving the therapeutic effects are applied to clinical use achieving certain good therapeutic effects.

Fructus piperis, a medicinal and edible plant, is a commonly used seasoning in the world. See “Chinese Pharmacopoeia” (2010), p227 and “Chinese Materia Medica” (vol. Mongolian medicine), p287. Fructus piperis is the dry, sub-mature or mature fruit of Piper nigrum L., a Piperaceae plant. It is harvested when the fruit color turns to dark green, which usually is from the end of autumn to next spring. Then the fruit was sun-dried to obtain Fructus piperis. The characteristics of Fructus piperis include: the spheric shape; 3.5-5 mm of diameter; pitchy surface with reticulated wrinkles on it, a top with the tiny style vestige and a base with a scar resulted from fruit axis abscission; hard in texture; peelable exocarp; gray or pale yellow endocarp; yellow-white, powdered in cross-section with small gaps therein; fragrant smells; and pungent taste. Property and flavor as well as meridian distribution: pungency and hot; belonging to stomach and large intestine channel. Function and indication: to warm (the middle warmer) and dispel cold, to subside chi, and to disperse phlegm. It is used for gastro-frigid vomiting, abdominal pain and diarrhea, anepithymia, as well as epilepsy and excessive phlegm.

“Ge Gengin” is a health-care food commercially available for lowering lipid (Approval number: hu wei shi zi 2002 No. 341), whose main components are alkaloids, especially piperine, extracted from Piperaceae plants Fructus Piperis Longi and Fructus Piperis etc.

Sinse 2002, Bo rijihangeriletu, Zhuang J in, Ruiguo Zhao, Laulan Bao, Yong Wu, Chunjie Ma, iingfen Han, et al. have dedicated to extensive researches on the ethanol extracts of Piperaceae plants Fructus Piperis Longi, and the derivatives thereof, and for the first time separated piperonyl amine natural products of sufficient quantity: Pipernonaline (C₂₁H₂₇NO₃), Piperlonguminine (C₁₆H₁₉NO₃) and Piperine (C₇H₁₉NO₃).

Our research group has researched for several years, and discovered a piperonyl amine natural product which is a pepper nigrtun extract for lowering blood lipid. See Chinese invention Patent Application, Patent No. ZL2004100966111.7 submitted by Bo rijihangeriletu, Na shengsang, Zhuang un, Ruiguo Zhao, et al.

### SUMMARY OF THE INVENTION

The inventors have surprisingly discovered by a large number of researches the excellent blood lipid lowering effect of potassium piperonyl pentadienoate.

The object of the present invention is to provide the use of potassium piperonyl pentadienoate for lowering blood lipid.

In the technical schemes of the present invention, potassium piperonyl pentadienoate or potassium piperonylate or GB-K refers to the compound with the following structure:

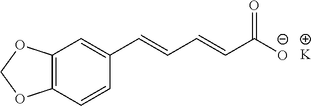 [SMILES: O=C([O-])/C=C/C=C/c1ccc2c(c1)OCO2 ; [K+]]

The present invention provides the use of potassium piperonyl pentadienoate in the preparation of a medicament for lowering blood lipid.

The present invention provides the use of potassium piperonyl pentadienoate in the preparation of a medicament for lowering serum total cholesterol.

The present invention provides the use of potassium piperonyl pentadienoate in the preparation of a medicament for lowering low density lipoprotein cholesterol.

The present invention provides the use of potassium piperonyl pentadienoate in the preparation of a medicament for preventing the decrease of high density lipoprotein cholesterol.

The present invention provides the use of potassium piperonyl pentadienoate in the preparation of a medicament for lowering triglyceride.

The present invention provides the use of potassium piperonyl pentadienoate in the preparation of a health-care product for lowering blood lipid.

The present invention provides the vise of potassium piperonyl pentadienoate in the preparation of a health-care product for lowering serum total cholesterol.

The present invention provides the use of potassium piperonyl pentadienoate in the preparation of a health-care product for lowering low density lipoprotein cholesterol.

The present invention provides the use of potassium piperonyl pentadienoate in the preparation of a health-care product for preventing the decrease of high density lipoprotein cholesterol.

The present invention provides use of potassium piperonyl pentadienoate in the preparation of a health-care product for lowering triglyceride.

The present invention provides the method for treating or preventing the diseases associated with the increased blood lipid in a subject in need thereof, comprising administering to the subject an effective amount of potassium piperonyl pentadienoate or the medicament comprising potassium piperonyl pentadienoate.

The present invention provides the method for treating or preventing the diseases associated with increased serum total cholesterol in a subject in need thereof, comprising administering to the subject an effective amount of potassium piperonyl pentadienoate or the medicament comprising potassium piperonyl pentadienoate.

The present invention provides the method for treating or preventing the diseases associated with increased low density lipoprotein cholesterol in a subject in need thereof, comprising administering to the subject an effective amount of potassium piperonyl pentadienoate or the medicament comprising potassium piperonyl pentadienoate.

The present invention provides the method for treating or preventing the diseases associated with the decrease of high density lipoprotein cholesterol in a subject in need thereof, comprising administering to the subject an effective amount of potassium piperonyl pentadienoate or the medicament comprising potassium piperonyl pentadienoate.

The present invent on provides the method for treating or preventing the diseases associated with increased triglyceride in a subject in need thereof, comprising administering to the subject an effective amount of potassium piperonyl pentadienoate or the medicament comprising potassium piperonyl pentadienoate.

The routes of administration for the potassium piperonyl pentadienoate according to the present invention may include oral or parenteral administration. The pharmaceutical composition or health-care food or product of the potassium piperonyl pentadienoate according to the present invention may, in addition to the active ingredient of potassium piperonyl pentadienoate, further include auxiliary material(s) which is/are pharmaceutically acceptable or acceptable in the health-care foods or products.

The oral drug formulations, injections or health-care foods or products prepared from the potassium piperonyl pentadienoate according to the present invention can be prepared and manufactured in accordance with common technologies or the prior art with respect to a person skilled in the art, such as those in “Pharmacy” Dafu Chui edits, 6^{TH}, People's Medical Publishing House, August 2007. The drug formulations, injections Of health-care foods or products prepared from the potassium piperonyl pentadienoate according to the present invention may also be formulated into liquid formulations (such as oral solutions, injections etc.) and solid formulations (such as tablets, capsules etc.). The daily dose of the potassium piperonyl pentadienoate according to the present invention may range from 10 mg to 5 g. The administration modes may be once per day or twice to three times per day.

The potassium piperonyl pentadienoate of the present invention may be prepared by the method described in Patent No. ZL20041009611.7: hydrolyzing a piperine (whose resource refers to UN 1330545A) with a solution of KOH in ethanol, to yield the potassium piperonyl pentadienoate. The reaction route is shown as follows:

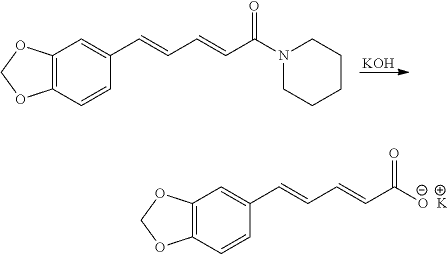

In accordance with teaching of the method for preparing the potassium piperonyl pentadienoate hereinabove, a non-cyclohexylamines piperonyl pentadienamide may be used as a starting material, and hydrolyzed with a solution of KOH in ethanol, to yield the potassium piperonyl pentadienoate.

The following unexpected effects are surprisingly discovered in the present invention by experiments: the potassium piperonyl pentadienoate is significantly superior to Simvastatin (control), and to piperonyl pentadienoic acid in lowering serum total cholesterol, triglyceride and low density lipoprotein cholesterol.

### BRIEF DESCRIPTION OF DRAWINGS

FIG. 1 shows 500 MHz ¹H-NMR spectrum for potassium piperonyl pentadienoate.

FIG. 2 shows 125 MHz ¹³C-NMR nuclear magnetic resonance spectrum for potassium piperonyl pentadienoate.

FIG. 3 shows the test results for prophylactic effects of piperonyl pentadiene cyclohexylamide (GB-O), piperonyl pentadienoic acid (GB-H), and potassium piperonyl pentadienoate (GB-K) on serum total cholesterol contents in white rats.

FIG. 4 shows the test results for prophylactic effects of GB-O, GB-H, and GB-K on serum triglyceride contents in white rats.

FIG. 5 shows the test results for prophylactic effects of GB-O, GB-H, and GB-K on serum low density lipoprotein cholesterol contents in white rats.

FIG. 6 shows the test results for prophylactic effects of GB-O, GB-H, and GB-K on serum high density lipoprotein cholesterol contents in white rats.

FIG. 7 shows the test results for therapeutic effects of GB-O, GB-H, and GB-K on serum total cholesterol contents in white rats.

FIG. 8 shows the test results for therapeutic effects of GB-O, GB-H, and GB-K on serum triglyceride contents in white rats.

FIG. 9 shows the test results for therapeutic effects of GB-O, GB-H, and GB-K on serum low density lipoprotein cholesterol contents in white rats.

FIG. 10 shows the test results for therapeutic effects of GB-O, GB-H, and GB-K on serum high density lipoprotein cholesterol contents in white rats.

### DETAILED DESCRIPTION OF THE INVENTION

The present invention is now further described in details with reference to the following examples. The following examples are illustrative and not for limiting the present invention.

### Example 1

### The Preparation of Potassium Piperonyl Pentadienoate (Gb-K)

The piperine (i.e. piperonyl pentadiene cyclohexylamide GB-O) and a certain amount of KOH were dissolved in ethanol. The mixture was refluxed for several hours at 80-90° C., and then standed still for cooling down for 12 hours, to yield hydrolyzate as a crystal. The crystal was washed with ethanol until the crystal became neutral, and then dried, under vacuum, to yield yellow solid, potassium piperonyl pentadienoate (GB-K). GB-K has a molecular formula. C₁₂H₉KO₄), EST-MS m/z: 217.0 [M-K].

Several experiments for the preparation under the specific reaction conditions are as follows:

| | Amount of | Aamount | | Reaction | Output of | Yield of |
|---|---|---|---|---|---|---|
| Piperine | ethanol | of KOH | Reaction | temperature | potassium salt | potassium salt |
| (g) | (ml) | (g) | time (h) | (° C.) | GB-K (g) | GB-K (%) |
| 1 | 7.8 | 1.77 | 8 | 86 | 0.764 | 85.05 |
| 2 | 15.6 | 3.54 | 8 | 86.5 | 1.637 | 91.12 |
| 5 | 39 | 8.85 | 8 | 86 | 4.020 | 89.51 |
| 10 | 78 | 17.7 | 8 | 85 | 8.006 | 89.13 |
| 20 | 156 | 35.4 | 8 | 84 | 15.96 | 88.84 |
| 40 | 312 | 70.8 | 8 | 84.5 | 31.56 | 87.84 |
| 80 | 624 | 141.6 | 8 | 84.5 | 65.12 | 90.62 |
| 150 | 1170 | 265.5 | 8 | 87.5 | 123.8 | 91.94 |
| 300 | 2340 | 531.0 | 8 | 87.5 | 248.0 | 92.01 |
| 2000 | 15600 | 3540 | 10 | 88 | 1687.3 | 93.92 |

The structure determination of potassium piperonyl pentadienoate (GB-K)

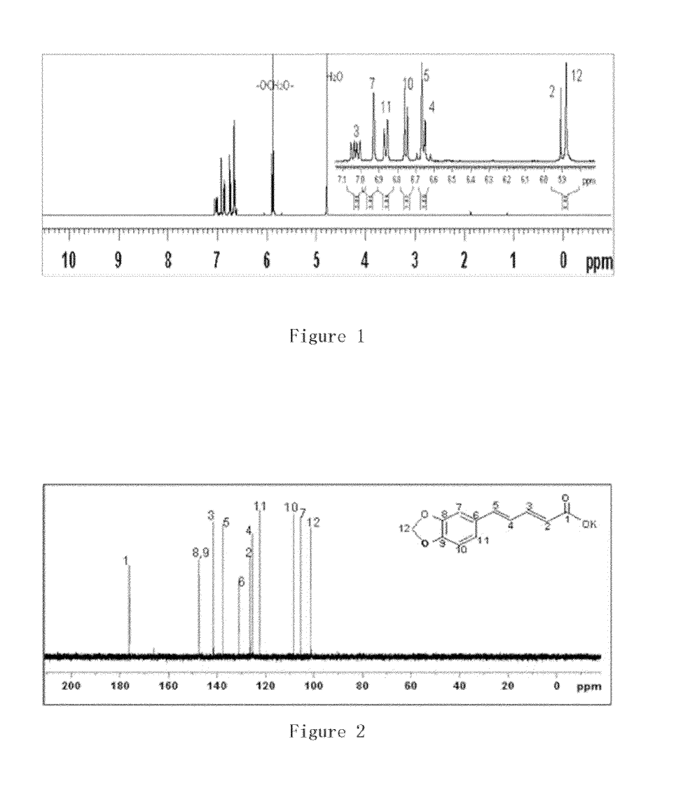

MHz ¹FI-NMR spectrum (see FIG. 1) and 125 MHz 13C-NMR nuclear magnetic resonnance spectrum (see FIG. 2) of potassium piperonyl pentadienoate were determined using a 500 MHz Bruker NMR spectrometer and deuterium oxide as a solvent.

Table of ¹H-NMR spectrum chemical shift data for potassium
piperonyl pentadienoate, unit (ppm)

| Hydrogen atom No. of | |
|---|---|
| GB-K | Chemical shift, ppm |
| H-2 | 5.914, 5.945 |
| H-3 | 7.045, 7.065, 7.075, 7.096 |
| H-4 | 6.534, 6.555, 6.565, 6.586 |
| H-5 | 6.612, 6.644 |
| H-7 | 6.831 |
| H-10 | 6.704, 6.721 |
| H-11 | 6.791, 6.807 |
| H-12 | 5.872 |

Table of ¹³C-NMR spectrum chemical shift data for potassium
piperonyl pentadienoate, unit (ppm)

| Carbon atom No. of | Chemical |
|---|---|
| GB-K | shift ppm |
| C-1 | 178.56 |
| C-2 | 129.25 |
| C-3 | 144.16 |
| C-4 | 127.98 |
| C-5 | 140.28 |
| C-6 | 133.61 |
| C-7 | 108.35 |
| C-8 | 150.11 |
| C-9 | 150.02 |
| C-10 | 125.22 |
| C-11 | 111.14 |
| C-12 | 103.91 |

### Example 2

### Comparative Experiments for Efficacy of Potassium Piperonyl Pentadienoate (GB-K) in Prophylactically Lowering Blood Lipid

(1) Experimental Materials

Experimental reagents: piperonyl pentadiene cyclohexylamide (GB-O), piperonyl pentadienoic acid (GB-H), potassium piperonyl pentadienoate (GB-K, Example 1), prepared by Inner Mongolia University.

Simvastatin (SVTT), manufactured by Zhejiang; Hisun Pharmaceutical Co. Ltd.; batch number: B1091138.

Total cholesterol kit, manufactured by BioSino BioTechnilogy & Science Inc.; batch number: 201009, Production Licence No.: Jing Yao Ran Sheng Chan Xu 20000271.

Triglyceride kit, manufactured by BioSino BioTechnilogy & Science Inc.; batch number: 201009, Production. Licence No.: Jing Yao Sian Sheng Chan Xu 20000271.

High density lipoprotein cholesterol kit, manufactured by BioSino BioTechnilogy & Science Inc.; batch number: 201007, Production Licence No.: Jing Yao Jian Sheng Chan Xu 20000271.

Low density lipoprotein cholesterol kit, manufactured by BioSino BioTechnilogy & Science Inc.; batch number: 201007, Production. Licence No.: Jing Yao Jian. Sheng Chan Xu 20000271.

Cholesterol, manufactured by Beijing Shuangxuan Microbe Culture Medium Products Factory; batch number: 20100619.

Sodium cholate, manufactured by Beijing Shuangxuan Microbe Culture Medium Products Factory; batch number: 0100525.

Experimental apparatus: PRONTO EVOLUTION AUTOMATIC ANALYZER (Italy); TDL-5 centrifuge (Shanghai Anting Scientific Instrument. Co. Ltd).

Experimental animal: Wistar male white rats, purchased from Vital River Laboratories.

High lipid feed formula: cholesterol 3%, lard 10%, sodium cholate 0.5%, basal feed: 865% (formula: flour 17.14%, rice flour 8.57%, maize 17.114%, bran 21%, fish flour 1.71%, dairy salt 0.857%, vitamin 0.085%, bean material 17.14%).

(2) Experimental Method

60 Wistar White male rats of 150±10 g of body weight were fed with common standard feed for 5 days, and then divided into 6 groups randomly. Each group included 10 rats. Group I was a blank group, where the rats were fed with common standard feed. Group II was a high lipid feed mode control group. Group III was a Simvastatin 10 mg/kg group. Group IV was a GB-O group. Group V was a GB-H group. Group VI was a GB-K group. High lipid feed mode control group were fed with high lipid feed together with distilled water. Groups III, IV, V, and VI were fed with high lipid feed together with required simvastatin, GB-O, GB-H, and GB-K suspensions (formulated by grinding with 0.5% sodium carboxymethycellulase CMC-Na) respectively. The dosing amount was 0.046 mmol/kg, and the amount of high lipid feed was 20 g per day for each rat. The grouping and the dosing amount for each group can be found in table 1.

TABLE 1
Grouping and the dosing amount for each group

| | Group | | | | | |
|---|---|---|---|---|---|---|
| | I | II | III | IV | V | VI |
| Test drug | Blank | High | Simvas- | GB-O | GB-H | GB-K |
| | | lipid | tatin | | | |
| Number of | 10 | 10 | 10 | 10 | 10 | 10 |
| animals | | | | | | |
| Dosing amount, | 0 | 0 | 0.0238 | 0.046 | 0.046 | 0.046 |
| mmol/kg | | | | | | |
| Dosing amount, | 0 | 0 | 10 | 13.1 | 10 | 11.7 |
| mg/kg | | | | | | |

(3) Observation, Determination and the Mathematical Methods of Statistics

The body weights of white rats were determined and recorded every day. The rats in each group were dosed basing on its body weight by feeding for 14 days at 22° C. of room temperature and 40-50% of relative humidity. On the 14^{th} day, after fasting and freely receiving water for 16 hours, its blood was sampled from aorta, placed for 30 min to blood coagulate, and centrifugated for 15 min at 3000 r/min to separate serum. The values of serum total cholesterol (TC), and triglyceride (TG) were determined on an automatic analyzer using enzymatic colorimetry, the value of high density lipoprotein cholesterol (HDL) was determined using a phosphotungstic acid-Mg precipitation method, and the value of low density lipoprotein cholesterol (LDL) was determined using the polyvinyl sulfate precipitation method.

All data were treated statistically, and were presented as means±standard deviations (x±s). The significance of difference was judged by T test. The calculation of p value was obtained by comparing the high lipid mode group with the blank control group, and comparing other test drug groups with the high lipid mode group. (p<0.01 represents extremely significant difference, p<0.05 represents significant difference, 0.05<p<1 represents having the tendency of action but no significant difference, p>1 represents counteraction).

(4) The Principle of Assays

The Assay of Total Cholesterol (TC) Enzymatic Colorimetry (CHOD-PAP).

The reaction temperature was 37° C. The cholesterol ester in serum was first hydrolyzed into free cholesterol (FC) and fatty acid with cholesterol ester hydrolase (CEH). Then, all FC in serum was oxidized into ch-4-en-3-one and H₂O₂ with cholesterol oxidase (COD). The resulting H₂O₂ was reacted with 4-aminoantipyrine (4-AAP) and 4-chlorophenol in the presence of peroxidase (POD) (Trinder reaction), to yield red quinine imine, which was calorimetrically tested at a 500 nm wavelength.

The Assay of Triglyceride (TC) by Enzymaticcolorimetry (GPO-PAP).

The reaction temperature was 37° C. The triglyceride ire serum was first hydrolyzed into glycerol and fatty acid with lipoprotein lipase (LPL). The resulting glycerol was reacted with ATP in the presence of glycerol kinase (GK), to produce glycerol-3-phosphate and ADP. The resulting glycerol-3-phosphate was oxidized into dihydroxyacetone phosphate and H₂O₂ with oxygen in the presence of glycerol phosphate oxidase (GPO). The resulting H₂O₂ was reacted with 4-aminoantipyrine (4-AAP) and 4-chlorophenol in the presence of peroxidase (POD) (Trinder reaction), to yield red quinine imine, which was colorimetrically determined at a 500 nm wavelength.

The Assay of Low Density Lipoprotein Cholesterol (LDL-C) by Polyvinyl Sulfate (PVS) Precipitation Method.

The LDL in serum was precipitated using PVS. The serum TC and the Ch in the resulting supernatant were determined using the enzymatic methods described hereinabove, and the LDL-C was calculated by subtracting the Ch from the serum TC. The polyethylene glycol monoether (PEGME) was added in a suitable amount in order to facilitate the precipitation of LDL.

The Assay of High Density Lipoprotein Cholesterol (HDL-C) Phosphotungstic Acid-Mg Precipitation Method.

After using phosphotungstic acid-Mg to precipitate low density lipoprotein (LDL) and very low density lipoprotein (VLDL) in serum, the resulting supernatant contained high density lipoprotein (HDL), and its cholesterol content was determined using the enzymatic method.

(5) The Test Data of Animal Experiment

The data of the tests for evaluating the effects of GB-O, GB-H, and GB-K on the blood lipid of high lipid mode white rats were listed in tables 2-5. The test results of serum total cholesterol values were shown in table 2. The test results of triglyceride values were shown in table 3. The test results of low density lipoprotein cholesterol values were shown in table 4. The test results of high density lipoprotein cholesterol values were shown in table 5.

TABLE 2
The serum total cholesterol (TC) contents in white rats on the 15^{th}
day after administration. (Unit mmol/L)

| | Group | | | | | |
|---|---|---|---|---|---|---|
| No. | I | II | III | IV | V | VI |
| 1 | 2.11 | 22.21 | 15.62 | 13.02 | 14.24 | 9.03 |
| 2 | 2.02 | 23.19 | 13.60 | 13.67 | 14.28 | 11.81 |
| 3 | 2.15 | 22.04 | 18.06 | 14.51 | 19.58 | 14.32 |
| 4 | 2.62 | 17.17 | 19.21 | 15.15 | 16.64 | 10.77 |
| 5 | 2.19 | 21.78 | 14.11 | 15.00 | 14.72 | 11.44 |
| 6 | 2.06 | 18.55 | 12.51 | 17.96 | 10.11 | 11.47 |
| 7 | 2.16 | 16.91 | 11.11 | 13.26 | 13.12 | 14.95 |
| 8 | 2.34 | 18.64 | 9.19 | 12.18 | 9.56 | 13.39 |
| 9 | 2.32 | 16.15 | 11.57 | 13.15 | 7.83 | 14.79 |
| 10 | 2.19 | 17.38 | 10.31 | 16.32 | 12.22 | 12.57 |
| Mean | 2.21 | 20.26 | 13.88 | 14.42 | 13.23 | 12.45 |
| Standard deviation | 0.17 | 2.63 | 3.27 | 1.74 | 3.49 | 1.91 |
| T test, in comparison | 5.5E−14 | — | 0.00034 | 9.423E−05 | 0.0003 | 2.46E−06 |
| with Group II | | | | | | |

TABLE 3
The serum triglyceride (TG) contents in white rats on the 15^{th} day
after administration. (Unit mmol/L)

| | Group | | | | | |
|---|---|---|---|---|---|---|
| No. | I | II | III | IV | V | VI |
| 1 | 1.33 | 4.73 | 1.23 | 0.84 | 1.23 | 1.38 |
| 2 | 1.01 | 1.77 | 1.31 | 1.10 | 1.70 | 1.04 |
| 3 | 1.94 | 2.05 | 1.28 | 0.75 | 1.30 | 1.04 |
| 4 | 1.47 | 2.01 | 1.12 | 1.14 | 1.15 | 0.99 |
| 5 | 1.78 | 1.63 | 1.53 | 1.21 | 1.64 | 0.87 |
| 6 | 1.21 | 5.02 | 0.93 | 1.02 | 1.71 | 0.97 |
| 7 | 1.68 | 1.60 | 1.36 | 0.96 | 0.91 | 1.03 |
| 8 | 1.33 | 1.52 | 1.26 | 1.16 | 0.88 | 1.36 |
| 9 | 1.59 | 2.3 | 1.00 | 1.12 | 1.06 | 1.16 |
| 10 | 1.13 | 1.92 | 1.19 | 0.98 | 1.58 | 1.00 |
| Mean | 1.44 | 2.68 | 1.22 | 1.02 | 1.31 | 1.08 |
| Standard | 0.29 | 1.50 | 0.18 | 0.15 | 0.32 | 0.16 |
| deviation | | | | | | |
| T test, in | 0.027 | — | 0.0080 | 0.0028 | 0.014 | 0.0038 |
| comparison with | | | | | | |
| Group II | | | | | | |

TABLE 4
The serum low density lipoprotein cholesterol (LDL) contents in
white rats on the 15^{th} day after administration. (Unit mmol/L)

| | Group | | | | | |
|---|---|---|---|---|---|---|
| No. | I | II | III | IV | V | VI |
| 1 | 1.71 | 3.89 | 3.37 | 3.05 | 4.86 | 2.00 |
| 2 | 1.12 | 6.51 | 3.63 | 3.10 | 3.51 | 3.17 |
| 3 | 1.62 | 5.52 | 2.93 | 8.24 | 5.29 | 1.77 |
| 4 | 1.70 | 3.21 | 5.81 | 7.93 | 4.53 | 2.72 |
| 5 | 1.65 | 4.15 | 4.98 | 4.07 | 5.85 | 2.80 |
| 6 | 1.47 | 4.81 | 5.32 | 6.67 | 4.24 | 5.92 |
| 7 | 1.41 | 3.54 | 3.73 | 3.10 | 2.74 | 2.57 |
| 8 | 1.48 | 5.06 | 5.14 | 2.98 | 4.19 | 5.28 |
| 9 | 1.58 | 9.37 | 3.99 | 5.60 | 2.68 | 3.83 |
| 10 | 1.57 | 5.10 | 3.33 | 5.42 | 5.09 | 1.93 |
| Mean | 1.53 | 4.51 | 4.22 | 5.01 | 4.29 | 3.19 |
| Standard | 0.17 | 1.17 | 0.99 | 2.06 | 1.05 | 1.41 |
| deviation | | | | | | |
| T test, in | 6.1E−06 | — | 0.18 | 0.90 | 0.22 | 0.016 |
| comparison | | | | | | |
| with Group | | | | | | |
| II | | | | | | |

TABLE 5
The serum high density lipoprotein cholesterol (HDL) contents in
white rats on the 15^{th} day after administration. (Unit mmol/L)

| | Group | | | | | |
|---|---|---|---|---|---|---|
| No. | I | II | III | IV | V | VI |
| 1 | 1.00 | 0.56 | 0.33 | 0.31 | 0.41 | 0.43 |
| 2 | 1.05 | 0.42 | 0.43 | 0.23 | 0.30 | 0.41 |
| 3 | 1.55 | 0.34 | 0.28 | 0.27 | 0.31 | 0.34 |
| 4 | 0.88 | 0.32 | 0.26 | 0.25 | 0.26 | 0.48 |
| 5 | 0.90 | 0.54 | 0.29 | 0.22 | 0.26 | 0.45 |
| 6 | 0.90 | 0.35 | 0.31 | 0.21 | 0.48 | 0.32 |
| 7 | 0.89 | 0.28 | 0.47 | 0.22 | 0.18 | 0.19 |
| 8 | 1.05 | 0.29 | 0.39 | 0.38 | 0.35 | 0.31 |
| 9 | 0.99 | 0.29 | 0.24 | 0.39 | 0.28 | 0.51 |
| 10 | 0.92 | 0.29 | 0.31 | 0.18 | 0.42 | 0.26 |
| Mean | 1.01 | 0.40 | 0.33 | 0.26 | 0.32 | 0.37 |
| Standard | 0.19 | 0.11 | 0.07 | 0.071 | 0.09 | 0.10 |
| deviation | | | | | | |
| T test, in | 4.1E−08 | — | 0.35 | 0.021 | 0.33 | 0.96 |
| comparison | | | | | | |
| with Group II | | | | | | |

(6) The Experimental Results and the Comparative Analysis for Blood Lipid Lowering

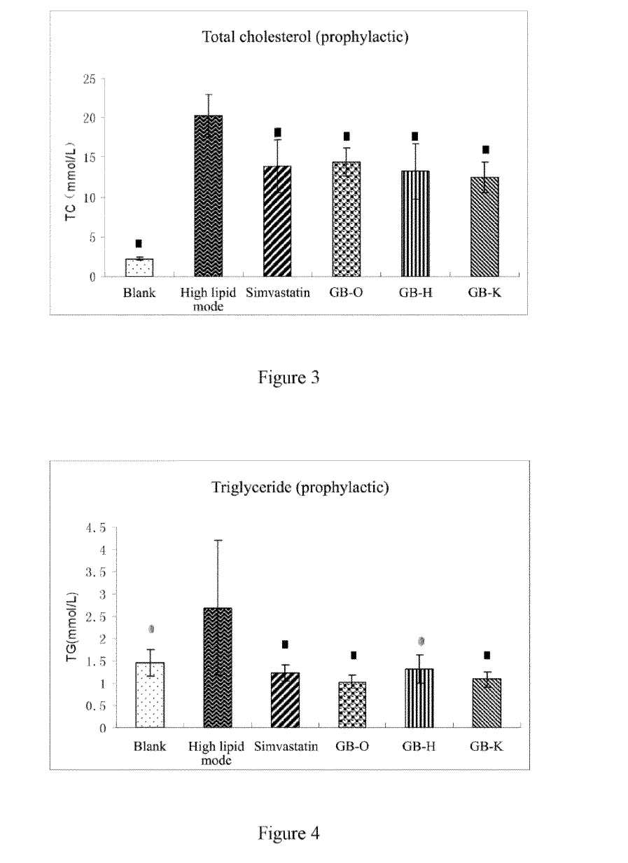

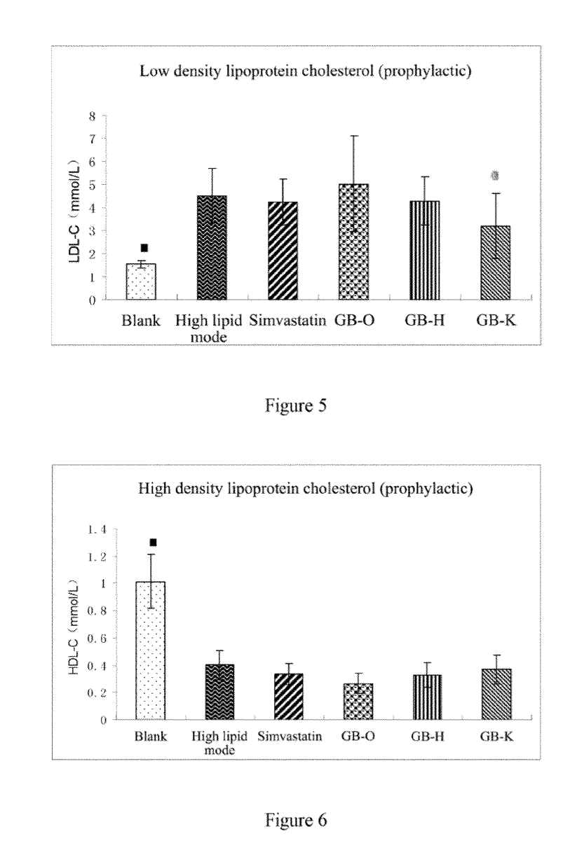

The data of the tests for evaluating the effects of GB-O, GB-H, and GB-K on the blood lipid of high lipid mode white rats were listed in tables 6-9. The test results of serum total cholesterol contents in white rats were shown in table 6 and FIG. 3. The test results of serum triglyceride contents in white rats were shown in table 7 and FIG. 4. The test results of low density lipoprotein cholesterol contents in white rats were shown in table 8 and FIG. 5. The test results of high density lipoprotein cholesterol contents in white rats were shown in table 9 and FIG. 6.

The Effects on Serum Total Cholesterol TC in White Rats:

In comparison with group II (high lipid mode control group), all groups show the effect of lowering total cholesterol. There are extremely significant differences for group III (Simvastatin 10 mg/kg), group IV (GB-O), group V GB-H), and group VI (GB-K).

TABLE 6
The analysis for test results of serum total cholesterol (TC) contents
in white rats.

| | Number of | | |
|---|---|---|---|
| Group | animals | Total cholesterol value | P value |
| I, blank group | 10 | 2.21 ± 0.17 | 5.5E−14 |
| II, high lipid mode | 10 | 20.26 ± 2.63 | — |
| group | | | |
| III, Simvastatin | 10 | 13.88 ± 3.27 | 0.0003 |
| IV, GB-O | 10 | 14.42 ± 1.74 | 9.42E−05 |
| V, GB-H | 10 | 13.23 ± 3.49 | 0.0003 |
| VI, GB-K | 10 | 12.45 ± 1.91 | 2.46E−06 |

Note:
P value refers to the comparison, between each group and group II: P < 0.05, significant difference; P < 0.01; extremely significant difference

The Effects on Serum Triglyceride TG in White Rats:

The serum TG in white rats demonstrates (see table 7 and FIG. 4) that each group, in comparison with group II (high lipid mode control group), shows the effect of lowering triglyceride. There is a significant difference for group V (GB-H), and extremely significant differences for group III. (Simvastatin 10 mg/kg), group IV (GB-O) and group VI (GB-K).

TABLE 7
The analysis for test results of serum triglyceride (TG) contents in
white rats. (unit mmol/L)

| | Number of | | |
|---|---|---|---|
| Group | animals | Triglyceride value | P value |
| I, blank group | 10 | 1.44 ± 0.2989 | 0.0279 |
| II, high lipid | 10 | 2.68 ± 1.5066 | — |
| mode group | | | |
| III, Simvastatin | 10 | 1.22 ± 0.1841 | 0.0080 |
| IV, GB-O | 10 | 1.02 ± 0.14770 | 0.0028 |
| V, GB-H | 10 | 1.31 ± 0.3219 | 0.0150 |
| VI, GB-K | 10 | 1.08 ± 0.1670 | 0.0038 |

Note:
P value refers to the comparison between each group and group II: P < 0.05, significant difference; P < 0.01, extremely significant difference

The Effects on Serum Low Density Lipoprotein Cholesterol LDL-C in White Rats:

The results of serum LDL-C in white rats demonstrate (see table 8 and FIG. 5) that group III (Simvastatin 10 mg/kg), group V (GB-H), and group VI (GB-K), in comparison with group II (high lipid mode control group), show the effect of lowering serum low density lipoprotein cholesterol value. There is a significant difference for group VI (GB-K).

TABLE 8
The analysis for test results of serum low density lipoprotein
cholesterol (LDL-C) contents in white rats. (unit mmol/L)

| | Number of | Low density | |
|---|---|---|---|
| Group | animals | Lipoprotein cholesterol | P value |
| I, blank, | 10 | 1.531 ± 0.1752 | 6.1E−06 |
| group | | | |
| II, high lipid | 10 | 4.518 ± 1.1729 | — |
| mode group | | | |
| III, Simvastatin | 10 | 4.223 ± 0.9989 | 0.1851 |
| IV, GB-O | 10 | 5.016 ± 2.0648 | 0.9091 |
| V, GB-H | 10 | 4.298 ± 1.0578 | 0.2295 |
| VI, GB-K | 10 | 3.199 ± 1.4145 | 0.0160 |

Note:
P value refers to the comparison between each group and group II: P < 0.05, significant difference; P < 0.01, extremely significant difference.

The Effects on Serum High Density Lipoprotein Cholesterol HDL-C in White Rats:

The results of HDL-C demonstrate see table 9 and FIG. 6) that group VI (GB-K), in comparison with group III (Simvastatin 10 mg/kg), group IV (GB-O), and group V (GB-H), show the effect of improving high density lipoprotein cholesterol.

TABLE 9
The analysis for test results of serum high density lipoprotein
cholesterol (HDL-C) contents in white rats. (unit mmol/L)

| | Number of | High density lipoprotein | |
|---|---|---|---|
| Group | animals | cholesterol | P value |
| I, blank group | 10 | 1.013 ± 0.1996 | 4.1E−08 |
| II, high lipid | 10 | 0.401 ± 0.1099 | — |
| mode group | | | |
| III, Simvastatin | 10 | 0.331 ± 0.0767 | 0.3458 |
| IV, GB-O | 10 | 0.266 ± 0.0719 | 0.0205 |
| V, GB-H | 10 | 0.325 ± 0.0902 | 0.3380 |
| VI, GB-K | 10 | 0.370 ± 0.1026 | 0.9660 |

Note:
P value refers to the comparison between each group and group II: P < 0.05, significant difference; P < 0.01, extremely significant difference

(7) Test Results and Conclusions

In the aspect of lowering total cholesterol, the test results demonstrate that there are extremely significant differences between GB-K, GB-O, GB-E groups and the high lipid mode control group, which demonstrates that GB-K can prevent the increase of total cholesterol, and GB-K is more significant in preventing the increase of total cholesterol.

In the aspect of lowering triglyceride, there are extremely significant differences between GB-K, GB-O, GB-1-1 groups and the high lipid mode control group. The effect for GB-K on preventing the increase of triglyceride is very significant.

In the aspect of lowering low density lipoprotein, there are extremely significant difference between GB-K group and the high lipid mode control group and GB-K shows the effect of decrease. GB-K shows more significant effect in preventing the increase of low density lipoprotein cholesterol, than GB-O, GB-H, and Simvastatin control groups.

In the aspect of preventing the decrease of high density lipoprotein cholesterol, GB-K group does not show significant increase in comparison with the high lipid mode control group, but shows the tendency of increase in comparison with GB-O, and GB-H groups.

In conclusion, GB-K possesses the effect on modulating blood lipid.

### Example 3

### Comparative Experiments of Efficacy for Potassium Piperonyl Pentadienoate (GB-K) in Therapeutically Lowering Blood Lipid

(1) Experimental Materials

Experimental reagents: piperonyl pentadiene cyclohexylamide (GB-O), piperonyl pentadienoic acid (GB-H), potassium piperonyl pentadienoate (GB-K, Example 1), prepared by Inner Mongolia University;

Simvastatin (SVTT), manufactured by Zhejiang Hisun Pharmaceutical Co. Ltd.; batch number: B1091138

Total cholesterol kit, manufactured by BioSino BioTechnilogy & Science Inc; batch number: 201009, Production Licence No: Jing Yao Jian Sheng Chan Xu 20000271

Triglyceride kit, manufactured by BioSino BioTechnilogy & Science Inc.; batch number: 201009, Production Licence No.: Jing Yao Jian Sheng Chan. Xu 20000271

High density lipoprotein cholesterol kit, manufactured by BioSino BioTechnilogy & Science Inc.; batch number: 201007. Production Licence No.: Jing Yao Jian Sheng Chan Xu 20000271

Low density lipoprotein cholesterol kit, manufactured by BioSino BioTechnilogy & Science Inc.; batch number: 201007, Production Licence No: Jing Yao hart Sheng Chan Xu 20000271

Cholesterol, manufactured by Beijing Shuangxuan Microbe Culture Medium Products Factory; batch number: 20100619

Sodium cholate, manufactured by Beijing Shuangxuan Microbe Culture Medium Products Factory; batch number: 0100525

Experimental apparatus: PRONTO EVOLUTION AUTOMATIC ANALYZER (Italy); IDL-5 centrithge (Shanghai Anting Scientific Instrument Co. Ltd.).

Experimental animal: Wistar male white rats, purchased from Vital River Laboratories.

High lipid feed formula: cholesterol 3%, lard 10%, sodium cholate 0.5%, basal feed: 86.5% (formula: flour 17.14%, rice flour 8.57%, maize 17.14%, bran 21%, fish flour 1.71%, dairy salt 0.857%, vitamin 0.085%, bean material 17.14%).

(2) Experimental Method

72 Wistar white male rats of 170±10 g of body weight were divided into 6 groups randomly and numbered. Each group included 12 rats. The rats were fed with common standard feed under experimental conditions and observed for 7 days, and its blood was sampled from tail for testing serum total cholesterol (TC). The test results can be seen in table 10. According to serum total cholesterol (TC) levels, the data for each group show that each index fell in the normal range. From the 8^{th} day, the group I was set as a blank group, and was fed with common standard feed. Other groups were fed with high lipid feed for 8 days in a row, and the blood was sampled from tail on the next day for testing TC. The test results can be seen in table 11. The data shows that the means of the indexes are higher than the normal values. P values from the comparison between high lipid mode value for each group and the normal value are all less than 0.05, which demonstrates that the mode of hyperlipoidemia is successfully prepared. The rats then were divided into 5 groups randomly (see table 12) according to TC levels. Each group included 10 rats (10 rats who were unsuccessful blood lipid modes were discarded). The rats were ted with high lipid teed continuously. Group was a high lipid feed mode control group. Group III was a Simvastatin 10 mg/kg group. Group IV was a GB-O group. Group V was a GB-H group. Group VI was a GB-K group. The high lipid feed mode control group was fed with high lipid feed together with distilled water. Groups III, IV, V, and VI were fed with high lipid feed together with required simvastatin, GB-O, GB-H, and GB-K suspensions (formulated by grinding with 0.5% sodium carboxymethycellulose CMC-Na) respectively. The dosing amount was 0.046 mmol/kg, and the amount of high lipid feed was 20 g per day for each rat. The grouping and the dosing amount for each group can be found in table 13.

TABLE 10
The serum total cholesterol (TC) contents in white rats on the 7^{th}
day after feeding with common feed. (Unit mmol/L)

Group

| No. | I | II | III | IV | V | VI |
|---|---|---|---|---|---|---|
| 1 | 2.07 | 2.01 | 2.26 | 2.35 | 1.92 | 1.96 |
| 2 | 2.11 | 2.35 | 2.38 | 2.08 | 2.03 | 1.97 |
| 3 | 2.06 | 2.41 | 2.61 | 2.07 | 2.35 | 2.23 |
| 4 | 1.97 | 2.31 | 2.41 | 2.07 | 2.46 | 2.05 |
| 5 | 1.93 | 1.92 | 2.31 | 2.23 | 1.99 | 2.07 |
| 6 | 2.02 | 1.83 | 2.05 | 2.35 | 2.38 | 2.38 |
| 7 | 2.20 | 1.97 | 1.97 | 2.61 | 2.03 | 2.04 |
| 8 | 2.19 | 2.07 | 1.92 | 2.41 | 2.56 | 1.83 |
| 9 | 1.93 | 2.01 | 1.83 | 2.06 | 1.94 | 1.85 |
| 10 | 1.89 | 2.27 | 2.09 | 2.08 | 2.07 | 2.37 |
| 11 | 2.26 | 2.26 | 1.94 | 2.19 | 2.03 | 2.33 |
| 12 | 2.22 | 2.31 | 2.42 | 2.15 | 2.22 | 2.61 |
| Mean | 2.07 | 2.14 | 2.18 | 2.22 | 2.16 | 2.14 |
| Standard | 0.12 | 0.19 | 0.24 | 0.17 | 0.21 | 0.24 |
| deviation | | | | | | |

TABLE 11
The serum total cholesterol (TC) contents in white rats on the 8^{th}
day after feeding with common feed. (Unit mmol/L)

| | Group | | | | | |
|---|---|---|---|---|---|---|
| No. | I | II | III | IV | V | VI |
| 1 | 2.36 | 13.67 | 13.25 | 13.68 | 15.24 | 12.41 |
| 2 | 2.33 | 12.41 | 13.71 | 14.71 | 13.62 | 12.52 |
| 3 | 2.05 | 12.87 | 18.06 | 15.02 | 13.57 | 12.22 |
| 4 | 1.97 | 14.77 | 17.05 | 9.38 | 10.28 | 13.87 |
| 5 | 2.67 | 15.24 | 13.64 | 12.44 | 11.41 | 13.45 |
| 6 | 2.22 | 13.03 | 12.57 | 12.71 | 14.91 | 11.06 |
| 7 | 2.03 | 12.38 | 12.11 | 13.33 | 12.82 | 10.99 |
| 8 | 2.19 | 14.51 | 9.37 | 12.18 | 12.46 | 10.72 |
| 9 | 1.99 | 13.45 | 11.57 | 13.15 | 9.77 | 10.24 |
| 10 | 2.37 | 17.05 | 10.31 | 15.27 | 12.22 | 10.26 |
| 11 | 2.07 | 18.24 | 13.64 | 13.24 | 12.41 | 11.74 |
| 12 | 2.31 | 16.34 | 18.26 | 15.07 | 11.44 | 9.37 |
| Mean | 2.21 | 14.49 | 13.62 | 13.34 | 12.51 | 11.57 |
| Standard | 0.20 | 1.90 | 2.85 | 1.64 | 1.66 | 1.36 |
| deviation | | | | | | |
| T test, in | — | 1.49E−16 | 2.57E−12 | 5.61E−17 | 3.81E−16 | 4.62E−17 |
| comparison | | | | | | |
| with Group I | | | | | | |

Note:
12 rats with underlines in the above table were excluded.

TABLE 12
Randomly grouping of rats according to TC levels (unit mmol/L)

Group

| No. | I | II | III | IV | V | VI |
|---|---|---|---|---|---|---|
| 1 | 2.36 | 13.67 | 13.25 | 13.68 | 18.06 | 12.41 |
| 2 | 2.33 | 12.41 | 12.38 | 14.71 | 13.67 | 12.52 |
| 3 | 2.05 | 12.87 | 15.24 | 11.67 | 16.34 | 12.22 |
| 4 | 1.97 | 14.77 | 17.05 | 12.41 | 11.44 | 13.87 |
| 5 | 2.22 | 15.24 | 13.64 | 12.44 | 11.41 | 13.45 |
| 6 | 2.03 | 13.03 | 12.57 | 12.71 | 14.91 | 11.06 |
| 7 | 2.19 | 13.71 | 12.11 | 13.33 | 12.82 | 15.27 |
| 8 | 1.99 | 14.51 | 13.24 | 12.18 | 12.46 | 13.57 |
| 9 | 2.07 | 13.45 | 11.57 | 13.15 | 11.74 | 13.64 |
| 10 | 2.31 | 15.02 | 15.07 | 18.24 | 12.22 | 18.26 |
| Mean | 2.15 | 13.46 | 13.61 | 13.45 | 13.50 | 13.62 |
| Standard | 0.14 | 1.23 | 1.69 | 1.88 | 2.25 | 1.98 |
| deviation | | | | | | |

TABLE 13
Grouping and dosing amount for each group

| | Group | | | | | |
|---|---|---|---|---|---|---|
| | I | II | III | IV | V | VI |
| Test drug | Blank | High | Simvas- | GB-O | GB-H | GB-K |
| | | lipid | tatin | | | |
| Number of | 10 | 10 | 10 | 10 | 10 | 10 |
| animals | | | | | | |
| Dosing amount, | 0 | 0 | 0.0238 | 0.046 | 0.046 | 0.046 |
| mmol/kg | | | | | | |
| Dosing amount, | 0 | 0 | 10 | 13.1 | 10 | 11.7 |
| mg/kg | | | | | | |

(3) Observation, Determination and the Mathematical Methods of Statistics

The body weights of white rats were determined and recorded every day. The rats for each group were dosed basing, on its body weight by feeding for 14 days at 22° C. of room temperature and 40-50% of relative humidity. On the 14^{th} day, after fasting and freely receiving water for 16 hours, the blood was sampled from aorta, placed for 30 min to blood coagulate, centrifugated for 15 min at 3000 r/min to separate serum. The values of serum total cholesterol (TC), and triglyceride (TG) were determined on an automatic analyzer using enzymatic colorimetry, the value of high density lipoprotein cholesterol (HDL) was determined using a phosphotungstic acid-Mg precipitation method, and the value of low density lipoprotein cholesterol (LDL) was determined using a polyvinyl sulfate precipitation method.

All data were treated statistically, and are presented as means standard deviations (x±s). The significance of difference was judged by T test. The calculation of p value was obtained by comparing the high lipid mode group with the blank control group, and comparing other test drug groups with the high lipid mode group. (p<0.01 represents extremely significant difference, p<0.05 represents significant difference, 0.05<p<1 represents having, the tendency of action but no significant difference, p>1 represents counteraction).

(4) The Principle of Assays

The Assay of Total Cholesterol (TC) by Enzymatic Colorimetry (CHOD-PAP).

The reaction temperature was 37° C. The cholesterol ester in serum was first hydrolyzed into free cholesterol (FC) and fatty acid with cholesterol ester hydrolase (CEH.) Then, all FC in serum was oxidized into ch-4-en-3-one and H₂O₂) with cholesterol oxidase (COD). The resulting H₂O₂, was reacted with 4-aminoantipyrine (4-AAP) and 4-chlorophenol in the presence of peroxidase (POD) (Trinder reaction), to yield red quinine imine, which was colarimetrically tested at 500 nm wavelength.

The Assay of Triglyceride (TG) by Enzymatic Colorimetry (GPO-PAP).

The reaction temperature was 37° C. The triglyceride in serum was first hydrolyzed into glycerol and fatty acid with lipoprotein lipase (LPL). The resulting glycerol was reacted with ATP in the presence of glycerol kinase (GK), to produce glycerol-3-phosphate and ADP. The resulting glycerol-3-phosphate was oxidized into dihydroxyacetone phosphate and H₂O₂ with oxygen in the presence of glycerol phosphate oxidase (GPO). The resulting H₂O₂ was reacted with 4-aminoantipyrine (4-AAP) and 4-chlorophenol in the presence of peroxidase (POD) (Trinder reaction), to yield red quinine imine, which was colorimetrically tested at 500 nm wavelength.

The Assay of Low Density Lipoprotein Cholesterol (LDL-C) by Polyvinyl Sulfate (PVS) Precipitation Method.

The LDL in serum was precipitated using PVS. The serum TC and the Ch in the resulting supernatant were determined using the enzymatic methods described hereinabove, and the LDL-C was calculated by subtracting the Ch from the serum. TC. The polyethylene glycol monoether (PEGME) was added in a suitable amount in order to facilitate the precipitation of LDL.

The Assay of High Density Lipoprotein Cholesterol (HDL-C) Phosphotungstic Acid-Mg Precipitation Method.

After using phosphotungstic acid-Mg to precipitate low density lipoprotein (LDL) and very low density lipoprotein (VLDL) in serum, the resulting supernatant contained high density lipoprotein (HDL), and its cholesterol content was determined using the enzymatic method.

(5) The Test Data of Animal Experiment

The data of the test for evaluating the effects of GB-O, GB-H, and GB-K on the blood lipid of hid) lipid mode white rats were listed in tables 14˜17. The test results of serum total cholesterol values were shown in table 14. The test results of triglyceride values were shown in table 15. The test results of low density lipoprotein cholesterol values were shown in table 16. The test results of high density lipoprotein cholesterol values were shown in table 17.

TABLE 14
The serum total cholesterol (TC) contents in white rats on the 15^{th}
day after administration. (Unit mmol/L)

| | Group | | | | | |
|---|---|---|---|---|---|---|
| No. | I | II | III | IV | V | VI |
| 1 | 2.42 | 15.37 | 9.37 | 11.57 | 11.28 | 9.36 |
| 2 | 2.37 | 16.27 | 8.61 | 13.14 | 9.36 | 11.33 |
| 3 | 2.16 | 16.37 | 11.34 | 10.67 | 15.24 | 9.25 |
| 4 | 2.06 | 13.51 | 7.61 | 12.34 | 12.34 | 8.92 |
| 5 | 2.19 | 14.62 | 9.38 | 10.04 | 9.37 | 11.45 |
| 6 | 1.99 | 15.27 | 8.81 | 9.71 | 12.03 | 9.32 |
| 7 | 2.34 | 13.81 | 8.39 | 12.06 | 10.25 | 8.37 |
| 8 | 2.09 | 16.08 | 12.67 | 11.01 | 10.37 | 13.57 |
| 9 | 2.17 | 13.54 | 10.34 | 12.34 | 9.36 | 9.38 |
| 10 | 2.42 | 15.37 | 9.37 | 11.57 | 11.28 | 9.36 |
| Mean | 2.22 | 15.02 | 9.59 | 11.45 | 11.09 | 10.03 |
| Standard | 0.16 | 1.10 | 1.50 | 1.09 | 1.82 | 1.59 |
| deviation | | | | | | |
| T test, in | 2.61E−18 | — | 2.96E−08 | 8.73E−07 | 1.57E−05 | 1.81E−07 |
| comparison with | | | | | | |
| Group II | | | | | | |

TABLE 15
The serum triglyceride (TG) contents in white rats on the 15^{th} day
after administration. (Unit mmol/L)

| | Group | | | | | |
|---|---|---|---|---|---|---|
| No. | I | II | III | IV | V | VI |
| 1 | 1.47 | 2.94 | 2.36 | 2.57 | 2.23 | 3.37 |
| 2 | 1.51 | 3.03 | 2.68 | 1.67 | 3.31 | 2.06 |
| 3 | 1.34 | 3.61 | 2.96 | 2.61 | 2.61 | 1.98 |
| 4 | 1.67 | 2.86 | 1.95 | 3.03 | 1.97 | 1.67 |
| 5 | 1.55 | 2.66 | 3.67 | 3.57 | 2.08 | 2.23 |
| 6 | 1.21 | 3.92 | 2.38 | 2.61 | 2.37 | 3.05 |
| 7 | 1.37 | 3.57 | 2.08 | 1.94 | 2.61 | 2.25 |
| 8 | 1.37 | 2.68 | 2.27 | 1.82 | 2.82 | 2.09 |
| 9 | 1.02 | 3.92 | 3.27 | 3.33 | 2.47 | 3.16 |
| 10 | 1.31 | 2.33 | 2.67 | 1.92 | 3.04 | 2.06 |
| Mean | 1.38 | 3.15 | 2.62 | 2.50 | 2.55 | 2.39 |
| Standard | 0.18 | 0.56 | 0.54 | 0.66 | 0.42 | 0.58 |
| deviation | | | | | | |
| T test, in | 2.1E−08 | — | 0.0482 | 0.0305 | 0.0145 | 0.0081 |
| comparison | | | | | | |
| with Group II | | | | | | |

TABLE 16
The serum low density lipoprotein cholesterol (LDL) contents in
white rats on the 15^{th} day after administration. (Unit mmol/L)

| | Group | | | | | |
|---|---|---|---|---|---|---|
| No. | I | II | III | IV | V | VI |
| 1 | 1.93 | 4.27 | 4.12 | 3.27 | 4.36 | 3.67 |
| 2 | 1.48 | 5.34 | 3.12 | 3.61 | 5.37 | 3.61 |
| 3 | 1.67 | 4.12 | 2.36 | 2.92 | 2.34 | 2.09 |
| 4 | 1.93 | 3.67 | 2.51 | 2.51 | 2.22 | 4.61 |
| 5 | 1.58 | 3.58 | 2.84 | 2.31 | 5.37 | 5.31 |
| 6 | 1.76 | 3.15 | 2.67 | 5.38 | 2.03 | 2.27 |
| 7 | 1.88 | 3.64 | 3.38 | 4.39 | 3.35 | 2.13 |
| 8 | 1.37 | 5.36 | 1.97 | 6.02 | 3.61 | 2.14 |
| 9 | 1.27 | 7.17 | 2.37 | 3.67 | 2.68 | 3.83 |
| 10 | 1.37 | 6.37 | 3.71 | 4.38 | 2.09 | 2.62 |
| Mean | 1.62 | 4.66 | 2.90 | 3.84 | 3.34 | 3.22 |
| Standard | 0.24 | 1.33 | 0.67 | 1.20 | 1.30 | 1.15 |
| deviation | | | | | | |
| T test, in | 1.34E−06 | — | 0.0016 | 0.1664 | 0.0377 | 0.0188 |
| comparison | | | | | | |
| with Group II | | | | | | |

TABLE 17
The serum high density lipoprotein cholesterol (HDL) contents in
white rats on the 15^{th} day after administration. (Unit mmol/L)

| | Group | | | | | |
|---|---|---|---|---|---|---|
| No. | I | II | III | IV | V | VI |
| 1 | 1.63 | 0.69 | 0.92 | 0.36 | 0.73 | 0.38 |
| 2 | 1.87 | 0.72 | 0.66 | 0.67 | 1.02 | 0.85 |
| 3 | 1.43 | 0.61 | 0.72 | 0.85 | 0.35 | 0.27 |
| 4 | 1.51 | 0.39 | 1.11 | 0.34 | 0.67 | 0.37 |
| 5 | 1.81 | 0.67 | 0.93 | 0.92 | 0.85 | 0.96 |
| 6 | 1.09 | 0.34 | 0.58 | 0.81 | 0.72 | 0.37 |
| 7 | 0.91 | 0.72 | 0.67 | 0.57 | 0.77 | 0.19 |
| 8 | 1.63 | 0.66 | 0.37 | 0.37 | 0.37 | 0.97 |
| 9 | 0.99 | 0.32 | 0.81 | 0.39 | 0.46 | 1.21 |
| 10 | 0.63 | 0.27 | 0.77 | 0.91 | 0.82 | 0.82 |
| Mean | 1.35 | 0.53 | 0.75 | 0.61 | 0.67 | 0.63 |
| Standard | 0.41 | 0.18 | 0.20 | 0.24 | 0.21 | 0.35 |
| deviation | | | | | | |
| T test, in | 2.58E−05 | — | 0.0245 | 0.4176 | 0.1473 | 0.4445 |
| comparison | | | | | | |
| with Group II | | | | | | |

(6) The Experimental Results and the Comparative Analysis for Blood Lipid Lowering

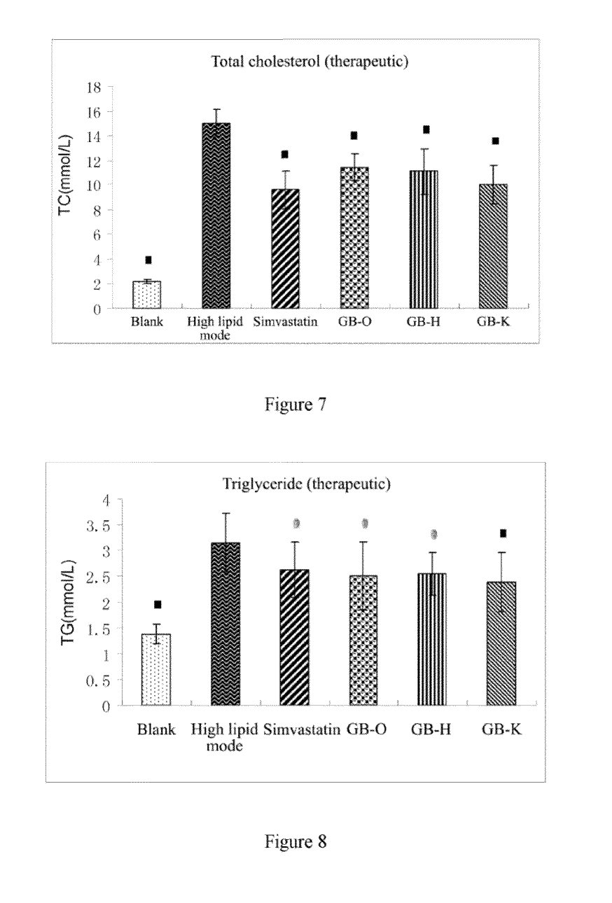

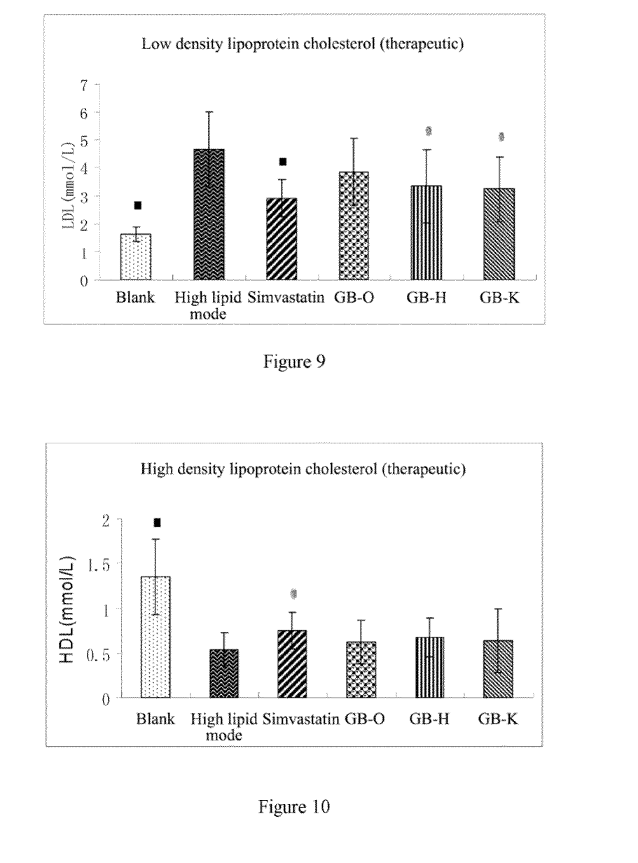

The results for evaluating the effects of GB-O, GB-H, and GB-K on the blood lipid of high lipid mode white rats were listed in tables 18-21 The analysis for test results of serum total cholesterol contents in white rats was shown in table 18 and FIG. 7. The analysis for test results of serum triglyceride, contents in white rats was shown in table 19 and FIG. 8. The analysis for test results of low density lipoprotein cholesterol contents in white rats was shown in table 20 and FIG. 9. The analysis for test results of high density lipoprotein cholesterol contents in white rats was shown in table 21 and FIG. 10.

The Effects on Serum Total Cholesterol TC in White Rats:

In comparison with group II (high lipid mode control group), all groups show the effects of lowering total cholesterol. There is extremely significant difference for group III (Sinwastatin 10 mg/kg), group IV (GB-O), group V (GB-H), and group IV (GB-K). Each group possesses the effect on lowering triglyceride.

TABLE 18
The analysis for test results of serum total cholesterol (TC) contents
in white rats.

| | Number of | Total cholesterol | |
|---|---|---|---|
| Group | animals | value | P value |
| I, blank group | 10 | 2.22 ± 0.16 | 2.61E−18 |
| II, high lipid mode | 10 | 15.02 ± 1.10 | — |
| group | | | |
| III, Simvastatin | 10 | 9.59 ± 1.50 | 2.96E−08 |
| IV, GB-O | 10 | 11.45 ± 1.09 | 8.73E−07 |
| V, GB-H | 10 | 11.09 ± 1.82 | 1.57E−05 |
| VI, GB-K | 10 | 10.03 ± 1.59 | 1.81E−07 |

Note:
P value refers to the comparison between each group and group II: P < 0.05, significant difference; P < 0.01, extremely significant difference.

The Effects on Serum Triglyceride TC in White Rats:

The serum TG in white rats demonstrates that each group, in comparison with group II (high lipid mode control group), shows the effect of lowering triglyceride. There are significant differences for group ill (Simvastatin 10 mg/kg), group TV (GB-O), and group V (GB-H), and a extremely significant difference for group VI (GB-K).

TABLE 19
The analysis for test results of serum triglyceride (TG) contents in
white rats. (unit mmol/L)

| | Number | | |
|---|---|---|---|
| Group | of animals | Triglyceride value | P value |
| I, blank group | 10 | 1.38 ± 0.18 | 2.1E−08 |
| II, high lipid | 10 | 3.15 ± 0.56 | — |
| mode group | | | |
| III, Simvastatin | 10 | 2.62 ± 0.54 | 0.0482 |
| IV, GB-O | 10 | 2.50 ± 0.66 | 0.0305 |
| V, GB-H | 10 | 2.55 ± 0.42 | 0.0145 |
| VI, GB-K | 10 | 2.39 ± 0.58 | 0.0081 |

Note:
P value refers to the comparison between each group and group II: P < 0.05, significant difference; P < 0.01, extremely significant difference.

The Effects on Serum Low Density Lipoprotein Cholesterol LDL-C in White Rats:

The results of SCRIM LDL-C in white rats demonstrate that, in comparison with group II (high lipid mode control group), group III (Simvastatin 10 mg/kg) shows a extremely significant difference, group IV (GB-O) shows the effect of lowering serum low density lipoprotein cholesterol value, and groups V (GB-H), and VI (GB-K) show significant differences.

TABLE 20
The analysis for test results of serum low density lipoprotein
cholesterol (LDL-C) contents in white rats. (unit mmol/L)

| | Number of | Low density | |
|---|---|---|---|
| Group | animals | lipoprotein cholesterol | P value |
| I, blank group | 10 | 1.62 ± 0.24 | 1.34E−06 |
| II, high lipid | 10 | 4.66 ± 1.33 | — |
| mode group | | | |
| III, Simvastatin | 10 | 2.90 ± 0.67 | 0.0061 |
| IV, GB-O | 10 | 3.84 ± 1.20 | 0.1664 |
| V, GB-H | 10 | 3.34 ± 1.30 | 0.0377 |
| VI, GB-K | 10 | 3.22 ± 1.15 | 0.0188 |

Note:
P value refers to the comparison between each group and group II: P < 0.05, significant difference; P < 0.01, extremely significant difference.

The Effects on Serum High Density Lipoprotein Cholesterol HDL-C in White Rats:

The results of HDL-C demonstrate that group ill (Simvastatin 10 mg/kg), group TV (GB-O) group V (GB-H), and group VI (GB-K), in comparison with group II (high lipid mode control group), show the effect of some extent, of increasing high density lipoprotein cholesterol, while Group III (Simvastatin 10 mg/kg) shows a significant difference.

TABLE 21
The analysis for test results of serum high density lipoprotein cholesterol
(HDL-C) contents in white rats. (unit mmol/L)

| | Number of | High density | |
|---|---|---|---|
| Group | animals | lipoprotein cholesterol | P value |
| I, blank group | 10 | 1.35 ± 0.41 | 2.58E−05 |
| II, high lipid | 10 | 0.53 ± 0.18 | — |
| mode group | | | |
| III, Simvastatin | 10 | 0.75 ± 0.20 | 0.0245 |
| IV, GB-O | 10 | 0.61 ± 0.24 | 0.4176 |
| V, GB-H | 10 | 0.67 ± 0.21 | 0.1473 |
| VI, GB-K | 10 | 0.63 ± 0.35 | 0.4445 |

Note:
P value refers to the comparison between each group and group II: P < 0.05, significant difference; P < 0.01, extremely significant difference.

(7) Test Results and Conclusions

In the aspect of lowering total cholesterol, the test results shows that there are extremely significant differences between GB-K, GB-O, GB-H groups and the high lipid mode control group, which demonstrates that they all can be used to treat the hyperlipoidemia induced by the increase of total cholesterol, and GB-K possesses more significant therapeutic efficacy.

In the aspect of lowering triglyceride, there are extremely significant differences between GB-K, GB-O, GB-H groups and the high lipid mode control group. The effect of GB-K on treating high triglyceride is very significant.

In the aspect of lowering low density lipoprotein, there is a significant difference between GB-K group and the high lipid mode control group, which demonstrates that GB-K can be used to treat the hyperlipoidemia induced by the increase of low density lipoprotein.

In the aspect of preventing the decrease of high density lipoprotein cholesterol, GB-K group does not show the significant difference, but shows the tendency of increase.

In conclusion, GB-K possesses the effect on modulating blood lipid.

In a word, the examples described above are preferred embodiments of the invention, but not used to limit the scope of the invention. Therefore, any modifications, equivalent substitutions and improvements made without departing from the spirits and principles of the invention shall all be included in the protection scope of the invention.

### INDUSTRIAL APPLICABILITY

The invention provides the use of potassium piperonyl pentadienoate in the preparation of medicaments or health-care products for modulating blood lipid.

The potassium piperonyl pentadienoate of the invention possesses the effects of lowering blood lipid, seam total cholesterol, triglyceride, and low density lipoprotein cholesterol, as well as the effect of preventing the decrease of high density lipoprotein cholesterol.

The potassium piperonyl pentadienoate of the invention are significantly superior to control drug Simvastatin, and also superior to piperonyl pentadienoic acid in lowering serum total cholesterol, triglyceride, and low density lipoprotein cholesterol.

1-21. (canceled)

22. A method for treating or preventing the diseases associated with the increased blood lipid, increased serum total cholesterol, increased low density lipoprotein cholesterol, the decrease of high density lipoprotein cholesterol or increased triglyceride, in a subject in need thereof, comprising administering to the subject an effective amount of potassium piperonyl pentadienoate or the medicament comprising potassium piperonyl pentadienoate.

23. The method according to claim 22, wherein the diseases are associated with the increased blood lipid.

24. The method according to claim 22, wherein the diseases are associated with the increased serum total cholesterol.

25. The method according to claim 22, wherein the diseases are associated with the increased low density lipoprotein cholesterol.

26. The method according to claim 22, wherein the diseases are associated with the decrease of high density lipoprotein cholesterol.

27. The method according to claim 22, wherein the diseases are associated with the increased triglyceride.
